(19) United States
(12) Patent Application Publication
(10) Pub. No.: US 2007/0245591 A1
(43) Pub. Date: Oct. 25, 2007

(54) Methods for drying objects using aerosols

(76) Inventors: Timothy H. Gens, (US); Jagjit S. Ratra, (US)

Correspondence Address: Timothy H. Gens, P.O. Box 61029, Palo Alto, CA, 94306, US

(21) Appl. No.: 11/408,562

(22) Filed: Apr. 22, 2006

(51) Int. Cl. F26B 3/00; A61K 9/12

(52) U.S. Cl. 34/443; 424/45

(57) ABSTRACT

The invention provides devices and methods of drying an object with a passive aerosol is created with no expenditure of energy from a drying liquid and delivered to the exposed surface of an object. The passive aerosol is created by rapidly decreasing the pressure of a carrier gas stream and entraining the drying liquid into the pressure means to create a venturi flow mixture of the carrier gas stream and the drying liquid. A primary baffle is positioned in the path of the venturi flow mixture so that the venturi flow mixture strikes against the primary baffle to create the passive aerosol. The passive aerosol can be used to create a drying layer through which the object is passed. The aerosol can also be used to apply directly to an exposed surface of an object to be dried

### CROSS-REFERENCE TO RELATED APPLICATIONS

This application is filed concurrently with an application entitled “Device and Methods For Drying Objects Using Aerosols” on the same date herewith.

### BACKGROUND OF THE INVENTION

1. Technical Field

The invention relates to drying manufactured objects using aerosols mechanically created by a venturi aerosol maker. More particularly, objects manufactured through the use of various processing liquids such as semiconductors, flat panel displays, optics, micro-electro-mechanical devices and other electronic devices are dried by the aerosol with little or no contamination of the object.

2. Background Information

It is well-known that the ability to perform a particle-neutral and water spot-free dry during surface preparation of various manufactured objects is a technology enabler. Such capability is especially important for electronic devices with high-aspect ratio features such as vias, deep trenches, contacts, and poly-Si features where water spots form easily at hydrophilic/hydrophobic interfaces. Spots that are large enough to overlap more than one die will result in defects, high leakage currents, critical-dimension variations, and film adhesion problems, all of which also may contribute to yield loss. The International Technology Roadmap for Semiconductors from SEMI set specifications for water spots at less than one per wafer at both the 130- and 100-nm nodes. The drying process must be implemented in a short time interval relative to other process steps and with the minimum use of energy and chemicals in an environmentally sensitive manner.

Known methods for drying integrated circuits use heated or superheated gases. For example, McConnell et al. in U.S. Pat. No. 4,911,761 et al. and Elsaway et al. in U.S. Pat. No. 6,328,809 disclose methods of drying semiconductor wafers by flowing a heated vapor past the wafers to be dried in a vessel. The preferred drying vapor is superheated isopropanol, which forms a minimum boiling azeotrope with water and is believed to displace water from the wafer surfaces. The vapor simultaneously flows in the vessel at one end and out the other end of the vessel. One of the drawbacks with these disclosed methods is that the drying vapor ineffectually flows out of the vessel. Accordingly, these methods use more than about half a liter of isopropanol for each standard drying cycle and this large quantity of isopropanol and other organic emissions must be captured and disposed off in an environmentally friendly manner. Another drawback is safely heating and handling the drying vapor which is flammable.

Other examples that similarly use heated or superheated gases are Bergman in U.S. Pat. No. 6,199,298B1 and Mertens et al. in U.S. Pat. No. 6,568,408 B2 which disclose methods of drying wafers by directing heated vapor to the wafer surface while rotating the wafer. Admittedly, the particle contamination performance is not equal to other known methods. Other drawbacks with these disclosed methods include the ineffectual delivery of the drying vapor, the difficulty in achieving condensation on the wafer surface, using large amounts of drying vapor, safely heating and handling a flammable drying vapor.

Another known example of drying integrated circuits is by using ultrasonically generated aerosols. Ferrell et al. in U.S. Pat. No. 6,270,584, No. 5,968,285, No. 5,964,958, No. 5,685,086, and No. 5,653,045 disclose methods and apparatus cleaning and/or drying objects in a vessel by atomizing isopropanol using an ultrasonic generator and a vibrating head. The vibrating head is mounted in the vessel directly over the object. As the rinse liquid is drained, some atomized particles settle onto the exposed surfaces of the objects, and displace and remove liquid residues from the exposed surfaces of the objects by a “chemical squeegeeing” effect. One of the drawbacks of these methods is the non-uniform, scattered distribution of relatively large droplets (a mass mean aerodynamic diameter significantly greater than about 50 microns and droplet diameters in excess of about 200 microns) dissolving into the rinse water while using an ultrasonic atomizer from Lechler GmbH in Germany to generate the reported test results. Another drawback of these methods is the large amount of isopropanol used in each standard dry cycle because a mask located underneath the vibrating head catches and disposes of the majority (about >90%) of the atomized isopropanol. Without the mask, the majority of the atomized isopropanol would uselessly dissolve into the rinse liquid or potentially carry large contamination particles onto the object. Another problem is reproducibility due to particle “spikes” caused by contamination particles collecting on the vibrating head and in the shunt leading to the vibrating head because they were not part of a loop, but instead a “dead leg”. When the ultrasonic generator was turned off at the end of a duty cycle, the isopropanol evaporated, potentially leaving contamination particles on the interior of the shunt and the vibrating head. These particles could dislodge when the duty cycle started and fall onto and contaminate the object in the vessel. Another drawback is that the energy used by ultrasonic atomizer heats the atomized isopropanol with resulting organic emissions and also potentially imparts a static charge on the object which attracts contamination.

The need remains for reproducibly drying objects without contamination while requiring no energy and only environmentally friendly amounts of a drying liquid. Preferably, the drying process is performable over a wide range of temperatures and is scalable to objects of different sizes and shapes. A few of the advantages of overcoming these problems is a reproducible drying process with higher yields, less consumable usage, and a more energy-efficient manufacturing process.

### BRIEF SUMMARY OF THE INVENTION

Briefly, the invention provides, in one embodiment, device for drying an object with an exposed surface to be dried. A passive aerosol is created from a drying liquid and delivered to the exposed surface of an object. The passive aerosol is created by rapidly decreasing the pressure of a carrier gas stream and entraining the drying liquid into the pressure means to create a venturi flow mixture of the carrier gas stream and the drying liquid. A primary baffle is positioned in the path of the venturi flow mixture so that the venturi flow mixture strikes against the primary baffle to create the passive aerosol.

Another device for drying the surface of an object includes a process chamber having a process liquid having a surface for submerging the object beneath. A passive aerosol is created from a drying liquid and delivered over the surface of the process liquid to form a drying layer of the passive aerosol on the surface of the process liquid. The drying layer is moved across the surface of the object.

Another device for drying the surface of an object includes a process chamber having a process fluid having a surface for submerging the object beneath. An aerosol is created from a drying liquid. The aerosol has a mass median aerodynamic diameter of 50 microns or less. The aerosol is delivered over the surface of the process liquid to form a drying layer of the aerosol on the surface of the process liquid. The drying layer is moved across the surface of the object.

A method of drying an object includes submerging an object having a object surface in a process liquid having a process liquid surface; creating a passive aerosol; exposing the process liquid surface to the passive aerosol of a drying liquid to form a drying layer on the process liquid surface; and moving the drying layer across the object surface.

Another method of drying an object includes submerging an object having a object surface in a process liquid having a process liquid surface; creating an aerosol having a mass mean aerodynamic diameter of less than 50 microns; exposing the process liquid surface to the aerosol to form a drying layer on the process liquid surface; and moving the drying liquid layer across the object surface.

Another method of drying an object includes submerging an object having a object surface in a process liquid having a process liquid surface; creating an aerosol of a drying liquid with no expenditure of energy; exposing the process liquid surface to the aerosol to form a drying liquid layer on the process liquid surface; and moving the drying liquid layer across the object surface.

A method of drying an object having an exposed surface in the invention includes creating an aerosol from a drying liquid; and delivering the aerosol to the exposed surface of the object. Furthermore, the method can include rotating the object about a central axis.

### BRIEF DESCRIPTION OF THE SEVERAL VIEWS OF THE DRAWING

These features and advantages of the invention, as well as others, will be apparent from the following description when read in conjunction with the accompanying drawings, wherein:

FIG. 1 is a partial schematic view illustrating a drying liquid management system and a aerosol management system and a partial cross-sectional side view of a Venture Aerosol Maker in accordance with the invention;

FIG. 2A is a side view with the interior in phantom of a gas jet provided by the invention;

FIG. 2B is a top perspective view of the gas jet shown in FIG. 2A;

FIG. 3A is a cross-sectional view of a drying liquid entrainment device along lines A-A in FIG. 3B;

FIG. 3B is a top perspective view of a drying liquid entrainment device of the invention;

FIG. 4A is a side view with the interior in phantom of a primary baffle provided by the invention;

FIG. 4B is a perspective view of the primary baffle shown in FIG. 4A;

FIG. 5A is a cross-sectional side view of the Venturi Aerosol Maker during an aerosol creation stage;

FIG. 5B is a cross-sectional side view of the Venture Aerosol Maker during a self-cleaning stage;

FIG. 6A is a partially exploded perspective view of a stand alone dryer embodiment of the invention;

FIG. 6B is a side view of the stand alone dryer embodiment shown in FIG. 6A;

FIG. 7A is a front view of an immersion bench with an integrated dryer embodiment of the invention;

FIG. 7B is a cross-sectional view of the integrated dryer embodiment along the lines A-A in FIG. 7A;

FIG. 8 is a cross-sectional side view of a spin processor with a single wafer dryer embodiment of the invention;

FIG. 9 is a cross-sectional side view of a spin processor with a dual-sided, single wafer dryer embodiment of the invention;

FIG. 10 is a cross-sectional side view of a spin processor with another embodiment of a single wafer dryer;

FIG. 11A is a cross-sectional side view of a vertical single wafer dryer embodiment of the invention;

FIG. 11B is a cross-sectional side view of the vertical single wafer dryer of FIG. 11A with the wafer being extracted through an aerosol and optional purge gas;

FIG. 12A is a cross-sectional side view of a dryer process chamber starting a drying cycle illustrating one embodiment of the inventive method;

FIG. 12B is a cross-sectional side view of a dryer process chamber in FIG. 11A during a controlled drain one embodiment of the inventive method;

FIG. 12C is a cross-sectional side view of a dryer process chamber in FIG. 11A with optional purge gas illustrating one embodiment of the inventive method;

FIG. 13 is a cross-sectional side view of a dryer process chamber illustrating another embodiment of the inventive method;

FIG. 14A is a schematic side view of a gas jet and entrainment device creating a venturi flow using one embodiment of the invention;

FIG. 14B is a schematic side view of a gas jet and entrainment device creating a venturi flow using another embodiment of the invention;

FIG. 15 is a flow chart of one embodiment of the inventive method;

FIG. 16 is a flow chart of a single object embodiment of the inventive method; and

FIG. 17 is a flow chart of another single object embodiment of the inventive method.

### DETAILED DESCRIPTION OF THE INVENTION

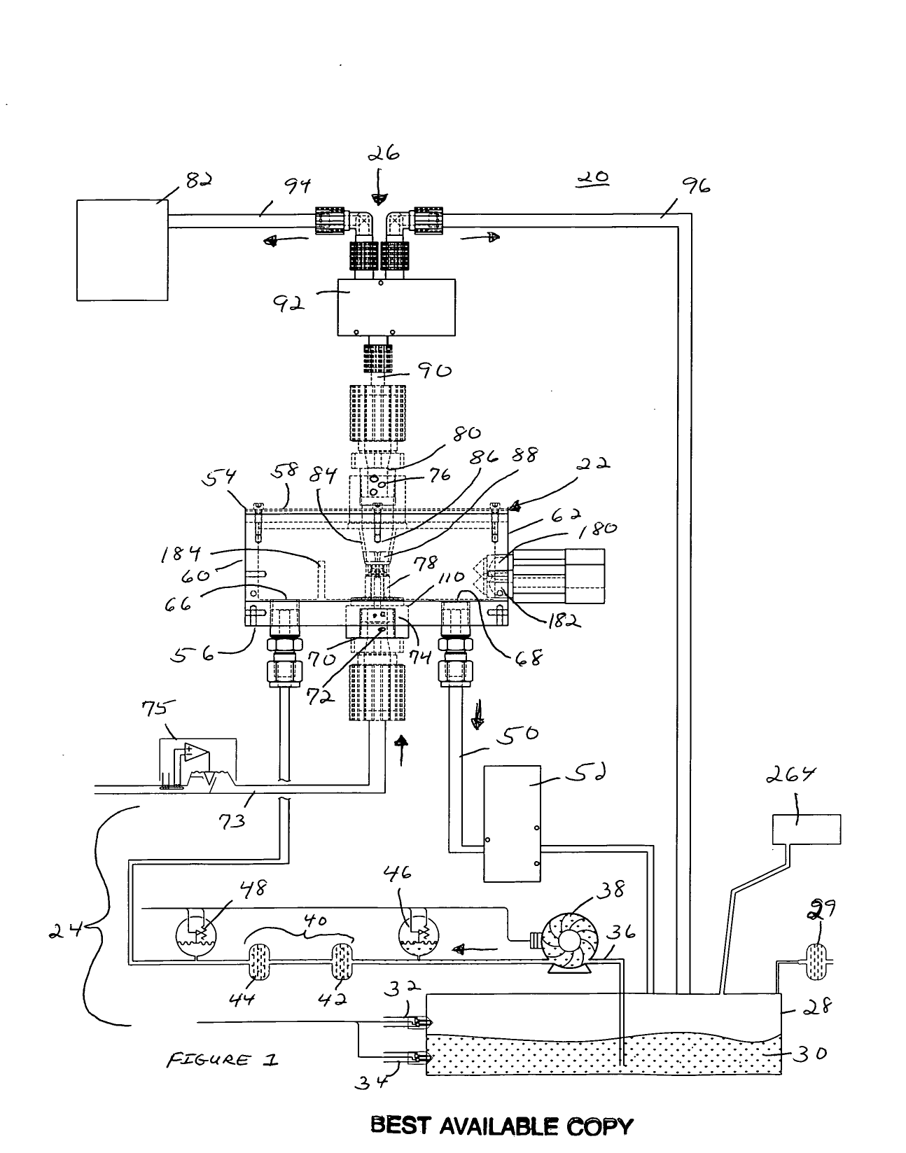

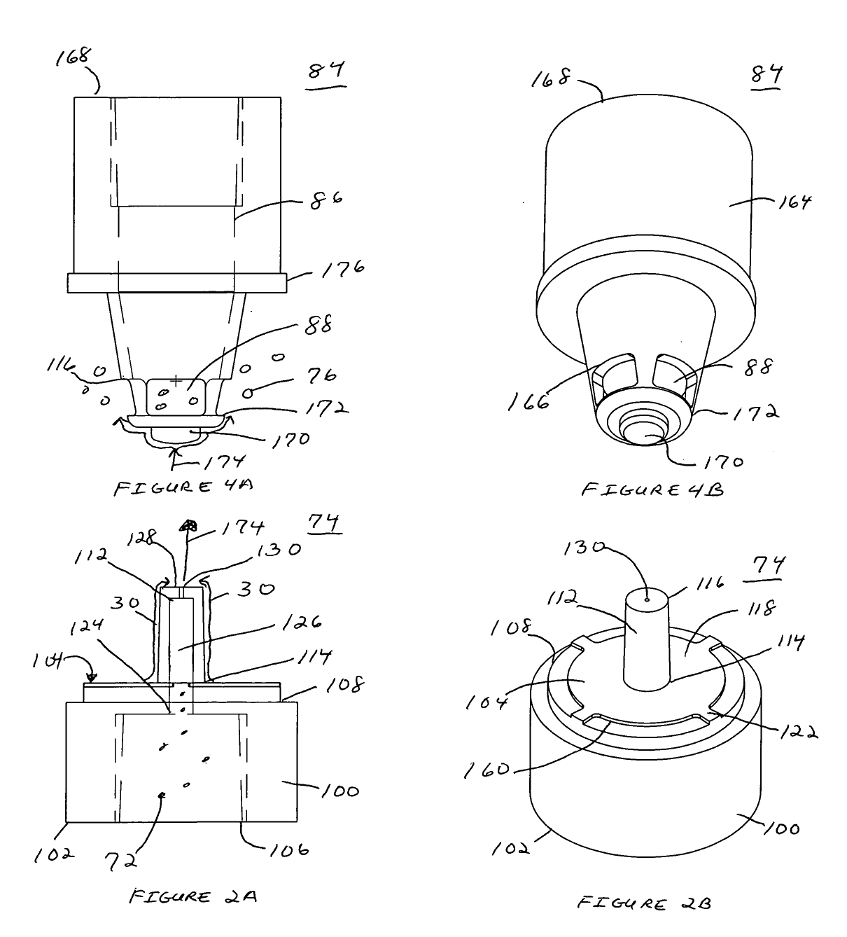

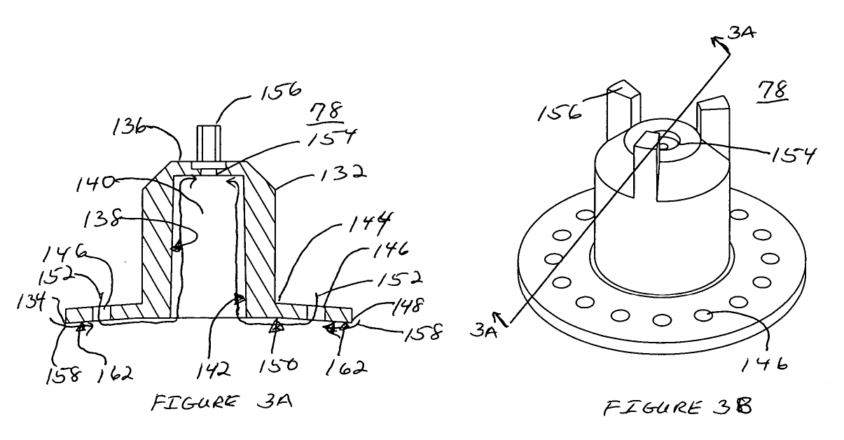

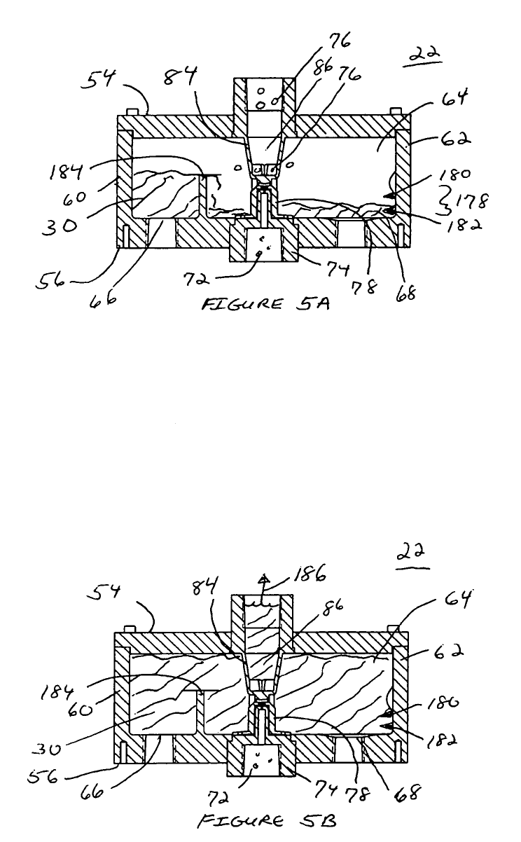

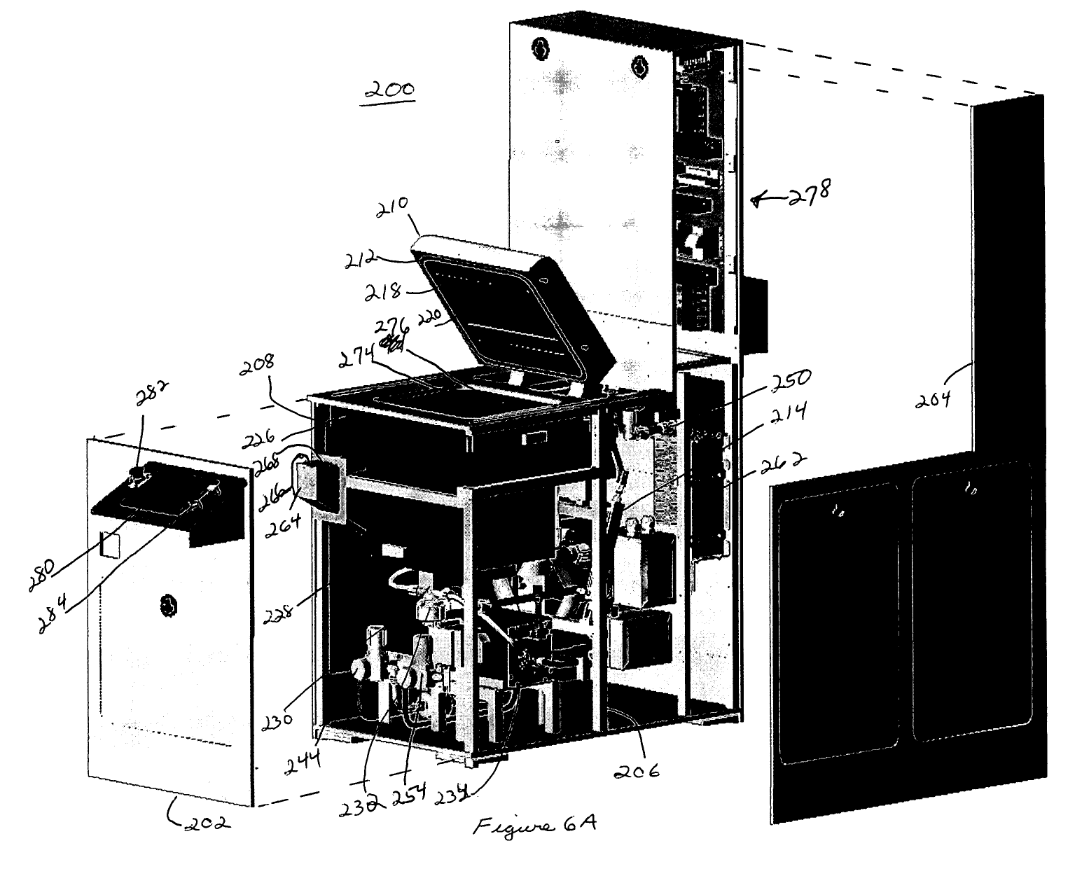

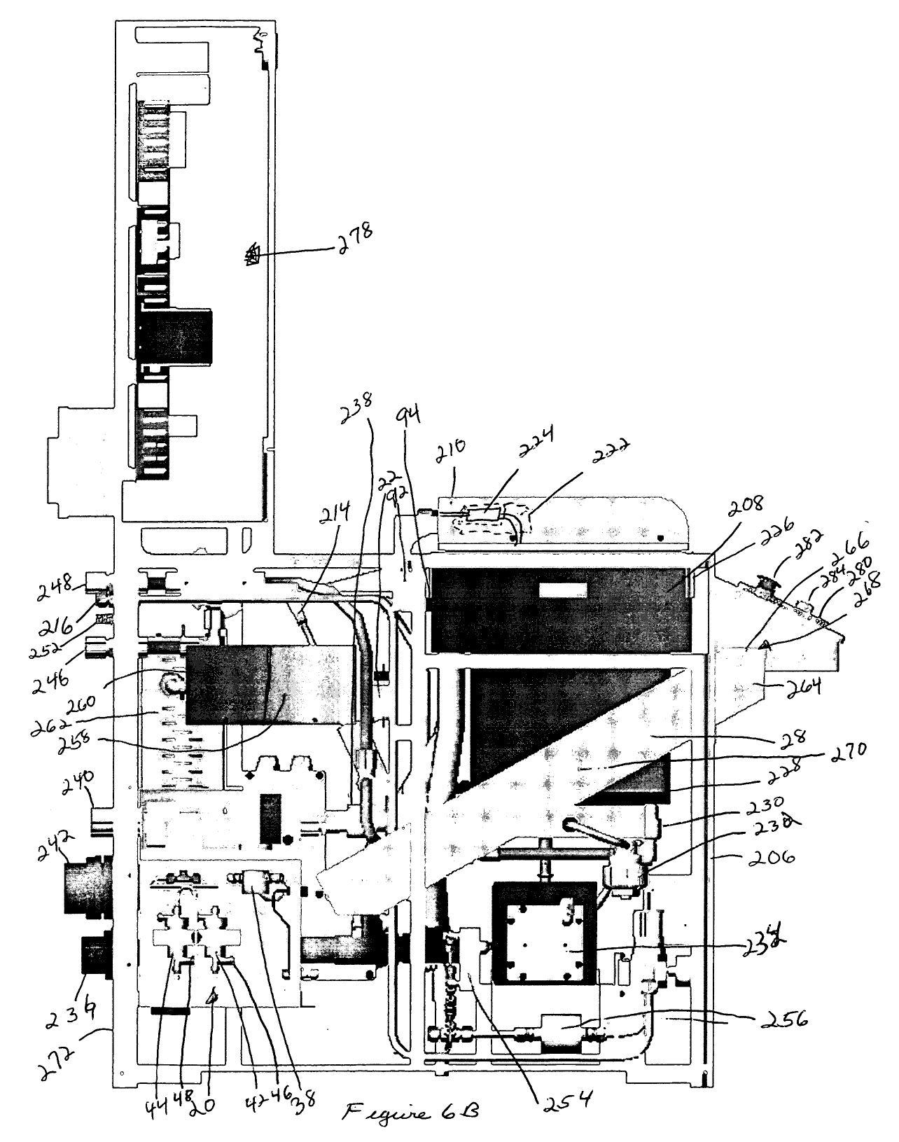

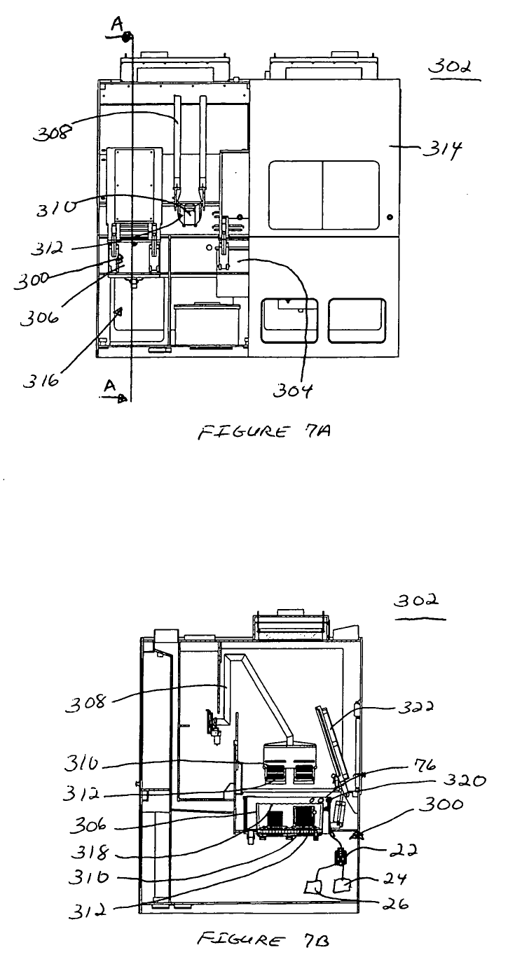

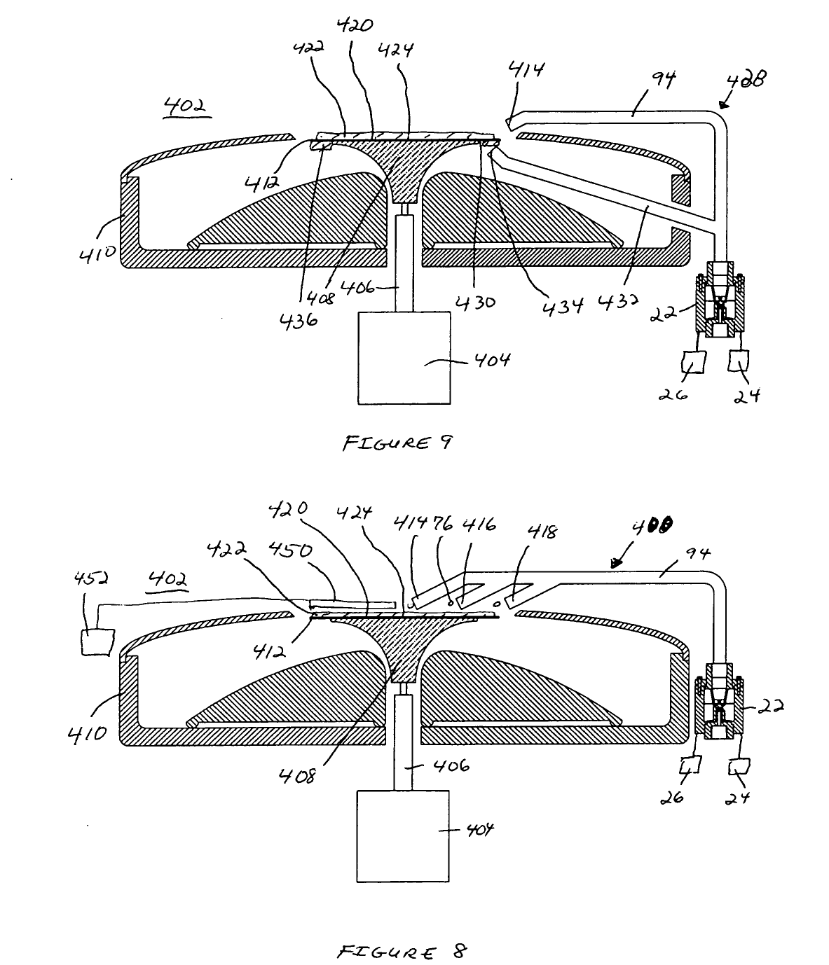

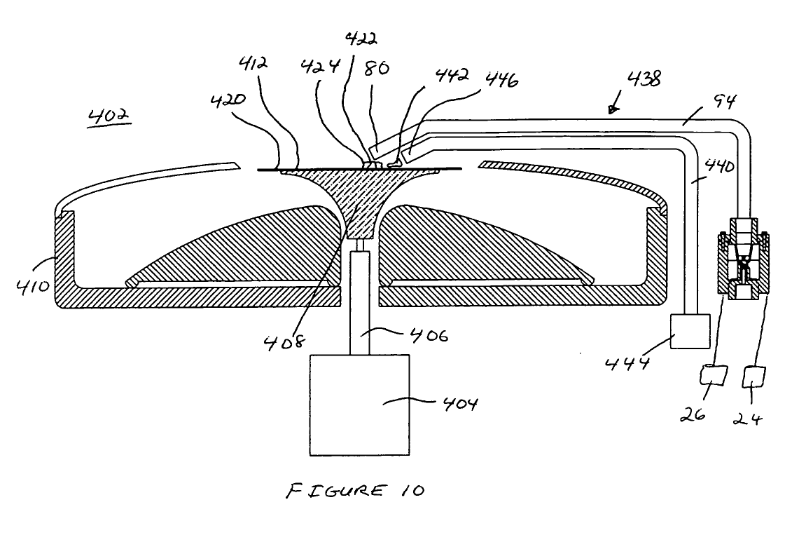

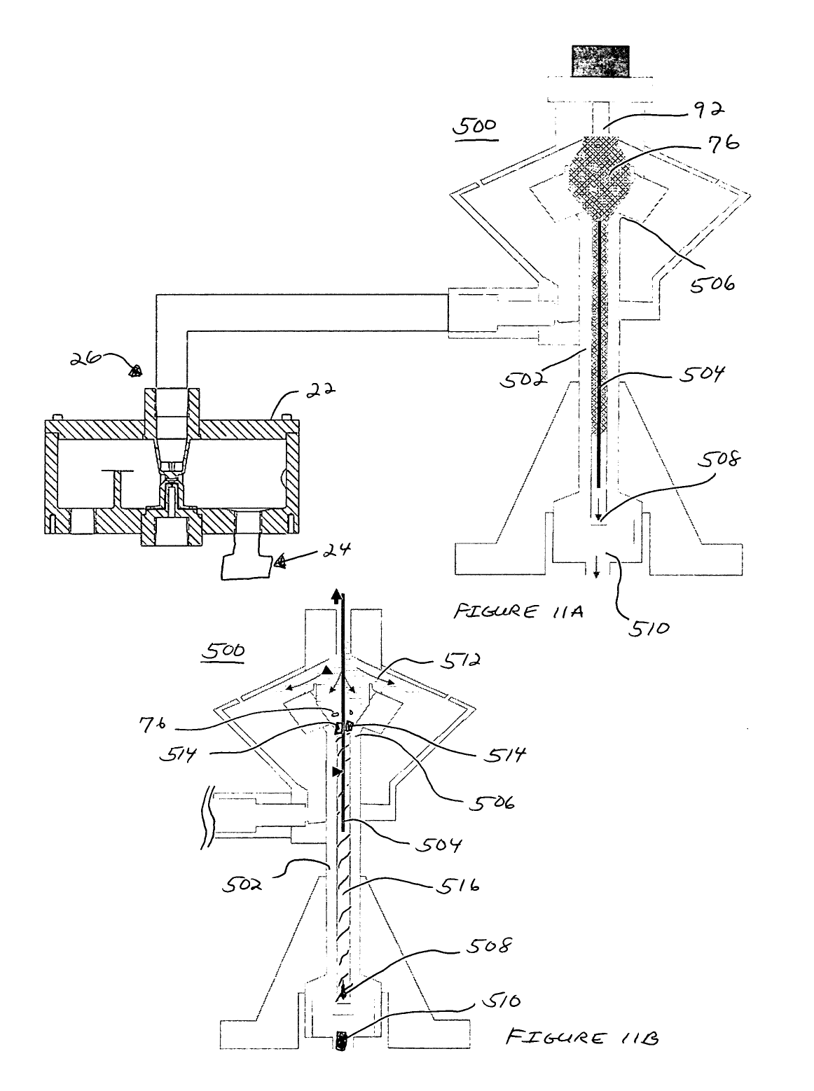

Referring now to FIG. 1, a self-cleaning system 20 in accordance with the invention includes a Venturi Aerosol Maker (VAM) 22, a drying liquid (DL) management system 24, and a aerosol management system 26. The DL management system 24 includes a container 28 for holding the DL 30. Preferably, a high DL level sensor 32 and a low DL level sensor 34 are positioned in the container 28 to respectively determine whether the container 28 is full or needs filling. The container 28 can be the original container from the DL supplier, connected to a DL bulk feed from the operator's facility, a freestanding container into which the DL 30 is manually filled, or other reservoir means. The illustrated container 28 attaches to a sealable DL fill port 264 and a filtered vent port 29. A DL feed line 36 extends from the container 28 to a DL pump 38 which can control the feed of the DL 30 from the container 28 to the remainder of the DL management system 24 and VAM 22.

The DL feed line 36 also passes through a filter means, generally depicted as 40, which can contain one or more individual filters or other means of removing contaminants from the DL 30. Preferably, a first filter 42 contains apertures sized to remove more coarse contaminants from the DL. A 0.1 micron diameter aperture in the first filter 42 and a second filter 44 containing apertures sized at a 0.05 micron diameter to remove more fine contaminates from the DL 30 is suitable to achieve the 100 nm particle test results recited herein. Filters with smaller apertures can be used to further improve the cleanliness of the DL 30.

Preferably, the DL feed line 36 also contains a first pressure transducer 46 for monitoring the pressure of the DL 30 in the DL feed line 36 before the filter means 40 and a second transducer 48 after the filter means 40. The first 46 and second 48 transducers allow monitoring of the pressure differential across the filter means 40 to determine if the filter means 40 is no longer operating effectively and is becoming clogged as the pressure differential increases. The pressure transducers 46, 48 also provide a safety mechanism to prevent the DL feed line 36 from experiencing pressure beyond its rated capability.

The DL management system 24 also includes a DL return line 50 extending from the VAM 22 to the container 28. Preferably, the DL return line 50 includes a valve 52 which can stop the flow of the DL 30 between the VAM 22 and the container 28.

The DL feed line 36 and the DL return line 50 can be made of any suitable material which is non-reactive with the DL. Preferably, Teflon tubing is used with a diameter of about 0.5 inch. The materials used in the construction of the DL pump 38, filter means 40 and the DL valve 52 should also be made of materials that are non-reactive with the DL 30 so that these components do not contribute contamination particles in the DL 30.

The VAM 22 includes a housing 54 having a bottom wall 56, a top wall 58, and side walls 60 and 62 to define a VAM chamber 64 which is sealed to prevent fluid leakage. The bottom wall 56 contains therethrough a DL feed port 66 and a DL return port 68 for the DL 30 to enter and exit the VAM chamber 64. The bottom wall 56 also includes a carrier gas feed port 70 therethrough to deliver a carrier gas 72 into the VAM chamber 64 from a carrier gas feed line 73 controlled by a carrier gas flow controller 75. Attached to the bottom wall 56 is a gas jet 74 which creates an area of low pressure with the carrier gas 72 to entrain the DL 30 to begin to form an aerosol 76. Connected to the gas jet 74 is a liquid entrainment device 78 which partially fits over the gas jet 74.

The top wall 58 includes an aerosol exit port 80 therethrough for delivery of the aerosol 76 to an object holder 82. Attached to the top wall 58 is a primary baffle 84 having an interior 86 which includes an aerosol distribution port 88. The primary baffle 84 is aligned over the top of the gas jet 74 and entrainment device 78. The aerosol 76 and carrier gas 72 exit the VAM 22 through the aerosol distribution port 88, the interior 86 of the primary baffle 84, and the aerosol exit port 80 to enter into an aerosol exit tube 90 leading to a three-way valve 92. The three-way valve 92 is also connected to an aerosol feed tube 94 that leads to the object holder 82 and a second DL return line 96 that connects to the container 28. The three-way valve 92 is part of the aerosol management system 26 and completes either a pathway from the VAM 22 to the object holder 82 or a pathway from the VAM 22 to the container 28.

The gas jet 74 is shown in more detail in FIGS. 2A and 2B. The gas jet 74 includes a jet body 100 having a generally cylindrical shape and with a bottom end 102 and a top end 104. The bottom end 102 is partially open to expose a fitting 106 which is adapted to accept connection to the carrier gas feed line 73. The top end 104 of the jet body 100 mounts approximately flush with the VAM chamber 64 along the bottom wall 56 of the VAM 22. A flange 108 along the perimeter of the top end 104 of the jet body 100 mounts to a reciprocal flange 110 (seen in FIG. 1) integrally formed in the bottom wall 56 of the VAM 22. The gas jet 74 may be mounted to the VAM 22 in a variety a ways such as press fitting flange 108 into reciprocal flange 110, with fasteners, or by integrally forming the gas jet 74 with the VAM bottom wall 56. A rod 112 having a bottom end 114 is integrally attached to the top end 104 of the jet body 100. The rod 112 includes a top end 116 which extends into the VAM chamber 64 when the gas jet 74 is mounted to the VAM 22. The top end 104 of the jet body 100 provides a shelf 118 for mating with the entrainment device 78. The shelf 118 has a one or more notches like 120 which partially extend inward from the perimeter of the jet body 100 towards the bottom end 114 of the rod 112. The depth of the notch 120 in the surface of the shelf 118 is only an indentation and is not sufficient to create a hole through the jet body 100. The notch 120 is also defined by a plurality of shelf arms like 122 that are the portion of the shelf 118 for mating and supporting the entrainment device 78.

The fitting 106 of the gas jet 74 extends along the interior of the jet body 100 to connect to a bottom end 124 of a jet chamber 126 for directing the carrier gas 72 from the fitting 106 to the jet chamber 126. The jet chamber 126 includes a top end 128 with a jet orifice 130 which directs the carrier gas 72 from the jet chamber 126 into the VAM 22. The jet chamber 126 has a diameter which is larger than the diameter of the jet orifice 130 so that the velocity of the carrier gas 72 is accelerated as the carrier gas 72 flows from the jet chamber 126 through the orifice 130 into the VAM 22. The relative diameter sizes of the jet chamber 126 and orifice 130 are dependent on several factors such as the feed pressure of the carrier gas 72, desired exit pressure of the carrier gas 72 through the orifice 130 and the like. Examples of suitable dimensions for the diameters of the jet chamber 126 and orifice 130 are 0.122 inch and 0.031 inch respectively.

The entrainment device 78 is shown in more detail in FIGS. 3A and 3B. The entrainment device 78 includes an entrainment body 132 having a generally cylindrical shape for mating over the jet body 100 of the gas jet 74. The entrainment body 132 has a bottom end 134, a top end 136, and an interior wall 138. The interior wall 138 defines a cavity 140 into which mates the rod 112 (FIGS. 2A and 2B) of the gas jet 74. The rod 112 is slightly smaller in diameter than the cavity 140 diameter leaving a DL feed gap 142 between the exterior surface of the rod 112 and the interior wall 138 of the cavity 140 so that DL can flow therebetween from the bottom end 134 to the top end 136 of the entrainment body 132.

The bottom end 134 includes a base 144 extending outwardly from the entrainment body 132. The base 144 has at least one DL feed port 146 extending therethrough. The base 144 has a concave shape so that only the perimeter 148 of the bottom end 134 contacts the shelf arms 122 of the gas jet 74 for support leaving a channel 150 between the bottom end 134 of the entrainment body 132 and the shelf 118 of the gas jet 74. The channel 150 extends from the DL feed port 146 to the DL feed gap 142 to form the primary DL source 152 for aerosol creation by allowing DL to flow from the VAM chamber 64 through the DL feed port 146, across the channel 150, to the DL feed gap 142, and to the top end 136 of the entrainment device 78 to a DL feed ring 154. The top end 136 of the entrainment body 132 also includes a plurality of posts like 156 is integrally formed with the top end 136 of the entrainment device 78 and is used to align the primary baffle 84 over the DL feed ring 154 and jet orifice 130.

Referring to FIGS. 2A and 3A, the DL feed ring 154 which is where the interior wall 138 terminates and the DL 30 of the primary DL source 152 exits the interior wall 138 of the entrainment device and is directed towards the carrier gas 72 exiting from the jet orifice 130 of the gas jet 74. The DL feed gap 142 terminates at the underside of the DL feed ring 154 and feeds the DL 30 into the carrier gas 72 flowing from the jet orifice 130 to create a venturi flow as indicated by arrow 174 comprising the DL 30 and carrier gas 72. The venturi flow 174 is created by the pressure drop of the carrier gas 72 leaving the jet orifice 130 which entrains the DL 30. The pressure drop is also responsible for drawing the DL 30 up the DL feed gap 142 from the VAM chamber 64.

Referring to FIGS. 2B and 3A, a secondary DL source 158 for aerosol creation is formed by allowing DL to flow from the VAM chamber 64 along the exterior surface of the notch 120 to a back side 160 of the notch 120. A secondary DL feed gap 162 is defined between the back side 160 of the notch and the portion of the base 144 near the perimeter 148 of the bottom end 134 of the entrainment body 132. The secondary DL feed gap 160 extends from the back side 160 to the channel 150. The secondary DL source 158 then allows the DL to flow from the back side 160, through the secondary DL feed gap 162, across the channel 150, to the DL feed gap 142, and to the top end 136 of the entrainment device 78 to the DL feed ring 154.

The use of the primary DL source 152 and secondary DL source 158 provides greater control over the creation of the aerosol 76 so that a wider range of particle size, size distribution, DL concentration, and the like, can be selected for use. As an example, and not for limitation, a suitable size for the DL feed gap 142 is about 0.027 inch, for the channel 150 is about 0.027 inch, for the secondary DL feed gap 162 is about 0.020 inch, for the diameter of the DL feed ring 154 is about 0.055 inch, and for the diameter of the DL feed port 146 is about 0.075 inch.

The primary baffle 84 is shown in more detail in FIGS. 4A and 4B. The primary baffle 84 includes a baffle body 164 having a generally cylindrical shape tapered towards a bottom end 166 and having a top end 168. The interior 86 of the primary baffle 84 is shown in phantom in FIG. 4A and is closed at the bottom end 166 by a baffle cap 170 having a baffle edge 172. The venturi flow 174 comprising the DL 30 and carrier gas 72 (shown in FIG. 2A) strikes the baffle cap 170 and continues flowing to the baffle edge 172 to create the aerosol 76. Located near the bottom end 166 of the primary baffle 84 is one or more aerosol distribution ports like 88 that open to the interior 86 of the baffle body 164. The aerosol distribution port 88 is positioned to be generally perpendicular to the surface of the baffle cap 170 and to be reached after the venturi flow 174 strikes the baffle cap 170. The aerosol 76 and carrier gas 72 exit the VAM 22 through the aerosol distribution port 88, the interior 86 of the primary baffle 84 and, as shown in FIG. 1, continues through the aerosol exit port 80 to enter into an aerosol exit tube 90 leading to a three-way valve 92.

The baffle body 164 includes a baffle flange 176 around its perimeter for mounting with the VAM chamber 64 along the top wall 58 of the VAM 22. The baffle flange 176 is located along the baffle body 164 allowing the baffle cap 170 and aerosol distribution port 88 to extend into the VAM chamber 64. The primary baffle 84 may be mounted to the VAM 22 in a variety a ways such as press fitting baffle flange 176, using fasteners, or by integrally forming the primary baffle 84 with the VAM top wall 58.

The venturi flow 174 interaction with the primary baffle 170 creates an aerosol 76 having smaller diameter particles with a smaller size distribution. Those particles which are too large coalesce into the DL 30 flowing through the VAM chamber 64. On the way to the object holder 82, the aerosol 76 travels through the three-way valve 92 which acts as a secondary baffle to further refine the size and distribution of the aerosol particles. Again, a small percentage (about 1-3%) of the aerosol particles coalesce and drain down the aerosol exit port 80 back into the DL 30 in the VAM chamber 64. Particle size is usually reported as mass median aerodynamic diameter (MMAD), which is the diameter around which the mass of the aerosol is equally divided. This characterizes the population of aerosol particles produced and since the volume of the particle is determined by the cube of the radius, most of the particles will be smaller than the MMAD. The MMAD can be measured by instruments like a phase doppler. A suitable MMAD for the aerosol 76 in the invention is about 50 microns or less. Preferably, the MMAD of the aerosol 76 used in the invention is about 10 microns or less.

It is believed that the primary factors determining aerosol particle size are carrier gas velocity and the ratio of liquid to gas flow. An increase in gas velocity decreases particle size, whereas an increase in the ratios of DL to gas flow increases particle size. Gas velocity affects the flow rates for both the gas and the DL.

Another unique feature and advantage of the invention is that the aerosol 76 is created without any energy expenditure. The aerosol is non-electrical, non-thermal, and neutral-charged. This “passive” aerosol has physical and chemical properties which provide significantly better performance than atomized DL aerosols, DL vapors, and the like. The passive aerosol is a suspension in a continuous gas phase of fine particles of liquid that have not undergone a phase transformation into a vapor. The invention prefers to create the aerosol at or near room temperature. No energy is imparted to the DL that would increase its temperature to change the DL into a vapor.

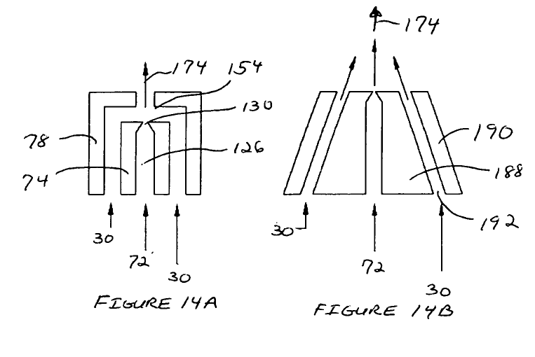

Referring to FIGS. 14A and 14B, another embodiment of the inventive VAM 22 is compared and described. FIG. 14A is a simplified version of the gas jet 74 and the entrainment device 78 illustrating how the DL 30 flows up the DL feed gap 142 to the underside of the DL feed ring 154. The carrier gas 72 flows through the jet chamber 126 and out the jet orifice 130 to entrain the DL 30 into the venturi flow 174. FIG. 14A illustrates creating a negative pressure with an internal entrainment of the DL 30 and carrier gas 72.

FIG. 14B is a simplified version of another embodiment of the invention with a different configuration for a gas jet 188, an entrainment device 190, and a DL feed gap 192 created therebetween. The carrier gas 72 flows out of the gas jet 188 to create a negative pressure with an external entrainment of the DL 30 to create the venturi flow 174. The invention includes other ways of creating negative pressure to entrain the DL into the carrier gas to create an aerosol.

Further operation of the self-cleaning system 20 is described by referring to FIGS. 1, 5A, and 5B. In FIG. 5A, the level of the DL 30 in the VAM chamber 64 is shown during the creation of the aerosol 76 by the VAM 22 or the aerosol creation stage. A VAM DL level sensing means, generally referred to as 178, controls the level of the DL 30 in the VAM chamber 64 so that there is sufficient DL 30 present to feed the primary DL source 152 and the secondary DL source 158 into the gas jet 74 and entrainment device 78. However, the DL 30 in the VAM chamber 64 should not be so high as to interfere with the DL 30 exiting from the DL feed ring 154 of the entrainment device 78 and the carrier gas 72 exiting from the jet orifice 130 of the gas jet 74. Preferably, a high VAM DL level sensor 180 and a low VAM DL level sensor 182 are positioned in the side wall 62 of the VAM to respectively determine whether the DL 30 level in the VAM chamber 60 is at the appropriate level or needs filling.

When the aerosol creation stage is to begin, the DL pump 38 stops pushing the DL 30 through the DL feed line 36 into the VAM chamber 64. The DL 30 level begins to drop as the DL 30 drains back to the container 28 through the DL return line 50. The DL valve 52 remains open as the DL 30 in the VAM chamber 64 drops to the desired level for aerosol creation to begin as indicated by the high VAM DL level sensor 180. The DL valve 52 can remain open as the DL pump 38 uses a low flow to keep the DL 30 level between the low VAM DL level sensor 182 and the high VAM Dl level sensor 180 during aerosol creation. Simultaneously, the three-way valve 92 opens the connection between the aerosol exit tube 90 and the aerosol feed tube 94 to provide a pathway for the aerosol 76 to reach the object holder 82 from the VAM 22. The connection to the secondary DL return line 96 is blocked by the three-way valve 92. The carrier gas 78 is also turned to high flow (between about 4 L/min to about 150 L/min) at the carrier gas feed port 70 and flows into the gas jet 74 as previously described. The DL 30 flows into the entrainment device 78 as previously described to begin the creation of the aerosol 76.

During to the aerosol creation step, the DL pump 38 runs at a low flow to advance sufficient DL 30 through the DL feed line 36 into the VAM chamber 64 to not only feed the aerosol creation but also to recirculate through the DL return line 50 to the container 28. Having the DL 30 continue to flow though the VAM chamber 64 during aerosol creation prevents any accumulation of contamination. Preferably, the VAM chamber 64 includes a turbulence wall 184 in FIG. 1 integrally formed with the bottom wall 56 and upstanding perpendicularly therefrom. The turbulence wall isolates the turbulence created by the DL pump 38 adding more DL 30 to the VAM chamber 64 during the aerosol creation stage. The turbulence wall allows the addition of more DL 30 to gently cascade over its top before coming in contact with the gas jet 74 and entrainment device 78.

Other suitable embodiments of the aerosol creation stage include, but are not limited to, closing the DL valve 52 during aerosol creation and running the DL pump 38 at a lower flow or pulsing flow to keep the DL 30 at an acceptable level. Another embodiment would fix the flow rate from the DL pump 38 and control the DL 30 level in the VAM chamber 64 with a proportional type valve for DL valve 52.

After the aerosol creation stage is complete, the self-cleaning system 20 can return to a cleaning stage. When the cleaning stage is to begin, the carrier gas 72 shifts to a low flow at the carrier gas feed port 70 sufficient to keep the DL 30 from entering the gas jet 72. The three-way valve 92 opens the connection between the aerosol exit tube 90 and the secondary DL return line 96 to provide a pathway for the DL 30 to flow to the container 28. The connection to the aerosol feed tube 94 is blocked by the three-way valve 92. The DL valve 52 is closed. The DL pump 38 shifts to a high flow and pushes the DL 30 from the container 28 through the DL feed line 36 to overfill the VAM chamber 64. As shown in FIG. 5B, the DL 30 level has filled the VAM chamber 64 and is proceeding up the aerosol exit tube 90 as indicated by arrow 186. As the DL pump 38 continues to a high flow feed of the DL 30 into the VAM 22, the DL 30 will fill the VAM 22, the aerosol exit tube 90, the three-way valve 92, and flow back to the container 28 through the secondary DL return line 96 for continued recirculation of the DL 30 through this loop.

Another embodiment of the cleaning stage creates a second recirculation loop by opening DL valve 52 and allowing the DL 30 to also exit from the VAM chamber 64 through the DL return line 50 and DL valve 52 to the container 28 for continued recirculation of the DL 30 through this second loop. Alternately, a valve could be used at the gas jet 74 to prevent the DL 30 from entering the gas jet 72.

The invention uses a unique multi-level flow rate method and device for circulating the DL 30 providing significantly increased cleanliness. A high flow rate circulates the DL 30 through the filter means 40 repeatedly during the cleaning stage which greatly improves the removal of particles. The resulting very pure DL 30 minimizes the possibility of contamination of the object from the DL 30. Using all of the same components in the self-cleaning system 20 and shifting to a low flow rate for the DL 30 during the aerosol creation stage is also minimizes particle contamination. Avoiding the use of different components when shifting between the cleaning stage and the aerosol creation stage eliminates possible sources of contamination. Other advantages of the high speed recirculation include, but are not limited to: eliminating down-time for the tool when the DL container is replenished, having the tool always ready to perform regardless of inactive periods, and the tool can be pressed into service faster. A suitable high flow rate is about 300 ml to 600 ml per minute which will typically circulate an entire gallon of the DL 30 through the filter means 40 in about two minutes. Of course this is an example and can be adjusted to be faster or slower by sizing the components like the DL pump 38 and the filter means 40 to the desired flow rate and resulting pressure.

The invention also uses a unique method and device for eliminating contamination from having the DL 30 evaporate on surfaces leaving behind particle contamination. Generally, the surfaces of all the components in the self-cleaning system 20 remain “wetted” by the DL 30 whether in the cleaning stage or in the aerosol creation stage. The DL 30 is not allowed to dry or evaporate on any surface so the opportunity for particle accumulation is eliminated and reproducible results are achieved. Specifically, when the VAM chamber 64 is flooded during the cleaning stage, all the surfaces of the gas jet 74, entrainment device 78, primary baffle 84, aerosol exit tube 90, and three-way valve 92 that were exposed to either the DL 30 or the aerosol 76 during the aerosol creation stage are submerged in DL 30 that is circulating through the filter means 40 during the cleaning stage.

One of the device embodiments of the invention is stand alone dryer 200 shown in FIGS. 6A and 6B. The dryer 200 includes a front panel 202 and a side panel 204 attached to a frame 206 for supporting an object holder, specifically a process chamber 208, sized to hold one or more of the objects (not shown) to be dried. A lid 210 includes a lid seal 212 for sealing the process chamber 208 when closed. The opening and closing of the lid 210 is controlled by an air cylinder 214 which receives compressed air from a facility feedstock (not shown) through a compressed air port 216 and an air regulator 244. The lid 210 contains one or more manifolds like 218 inserted on the underside 220 of the lid facing the process chamber 208. The manifold 218 directs an inert purge gas into the process chamber 208. The lid 210 has an interior 222 shown in phantom specifically in FIG. 6B which contains a purge gas filter 224 for minimizing particle contamination of the purge gas before it enters the process chamber 208.

The process chamber 208 has a top end 226 and a bottom end 228. Connected to the process chamber 208 near the bottom end 228 is a drain 230 having a drain valve 232 and a drain motor 234 which leads to a drain port 236 in the facility. The drain motor 234 variably controls the drain rate of any process liquid from the process chamber 208. The drain 230 is also connected to an air amplifier 238 and exhaust ports 240, 242. A process liquid feed port 246 and process liquid return port 248 directs one or more process liquids into and from the process chamber 208 through a process liquid valve 250.

A purge gas port 252 connects to the facility feedstock and delivers the purge gas through a purge gas regulator 254 and a purge gas primary filter 256. The purge gas is then split into two pathways. A first pathway leads to a VAM mass flow controller 258 which controls the delivery of the purge gas to the carrier gas feed port 70 and the gas jet 74 wherein the purge gas is used as the carrier gas 72 (as shown in FIG. 1 et al.). A second pathway leads the purge gas through a purge mass flow controller 260 into a heater 262 connecting with the filter 224 and the manifold 218 to direct the purge gas into the process chamber 208.

The container 28 for storing the DL has a unique construction integrating a DL fill port 264 that protrudes from the front panel 202 to allow easy accessibility for replenishing the DL in the container 28. The DL fill port 264 has a DL fill cap 266 which reversibly seals and extends upwardly so as to present a flat filling surface 268 that is generally level to the ground to minimize spillage when the DL is being poured into the DL fill port 264. The container 28 has an elongated body 270 which extends from the front panel 202 downwardly towards a back panel 272 of the dryer. The integration of the elongated body 270 with the DL fill port 264 provides a larger capacity for storing the DL and the downward slant allows gravity to aid in filling and dispensing the DL to the self-cleaning system 20. The container 28 connects to the DL pump 38, the first filter 42, the second filter, the first transducer 46, and the second transducer 48. The self cleaning system 20 also includes the VAM 22, the three-way valve 92 and the aerosol feed tube 94 that leads into an aerosol chamber port 276 in a side wall 274 of the process chamber 208. The aerosol chamber port 276 is in a position on the side wall 274 located above the highest level maintained by the process liquid in the process chamber 208.

The dryer 200 also includes an electrical cabinet generally depicted as 278. The front panel 202 includes a touch screen 280 with a stop button 282 and a start button 284 for operator control.

The DL should be inert or non-reactive with the object being dried and the other materials that the DL comes in contact with during the drying process. The DL should have a significantly lower surface tension than the process liquid or rinse liquid that is being removed. Preferably, the DL should have a surface tension of less than about 25 dynes/cm at 20 C.°. Examples of suitable Drying Liquids include isopropanol, methanol, ethanol, acetone, tetrahydrofuran, perfluorohexane, ether, hexane, hydrofluororether (methyl nonafluorobutyl ether or methoxy nonafluorobutane collectively referred to herein as HFE), and an ethylated hydrofluoroether commercially available from the 3M Company in Minneapolis, Minn. The DL can also be a combination of these examples.

The carrier gas should also be inert or non-reactive with the object being dried and the other materials that the DL comes in contact with during the drying process. Examples of a suitable carrier gas are nitrogen or carbon dioxide.

The process liquid should have a significantly high surface tension than the DL. An example of a suitable process liquid is de-ionized water, but also can include various chemistries that are aqueous based or mixed with water.

Examples of suitable materials for the process chamber include polyvinylidene fluoride, polypropylene, quartz, Halar, or stainless steel coated with Teflon. Suitable materials for the manifold minimize contamination of the purge gas and are heat resistant should the purge gas be heated. An example of a suitable material is a linear aromatic polymer called PEEK which comprises oxy-1,4-phenyleneoxy-1,4-phenylene-carbonyl-1,4-phenylene.

Another of the device embodiments of the invention is an integrated dryer 300 which is part of an immersion bench 302, wet bench, process line, or the like, described in more detail in FIGS. 7A and 7B. The typical immersion bench 302 includes a plurality of process chambers like 304 and 306 which can be serviced by a handling robot 308 for moving the objects, in this instance a plurality of semiconductor wafers 310 stacked in a cassette 312, from one process chamber 304 to the next process chamber 306. The robot 308 can insert and remove the wafers 310 into and from each process chamber like 306. Alternately, the wafers 310 can be handled manually. A door 314 typically isolates the environment around the process chambers 304, 306.

The immersion bench 302 provides an envelope 316 for generally placing the integrated dryer 300 including the previously described VAM 22, the DL management system 24, and aerosol management system 26 with many of the same components also previously described above with the stand alone dryer 200. Accordingly, FIG. 7B specifically depicts the process chamber 306 with the plurality of wafers 310 supported in the cassette 312 located therein and submerged in a process liquid 318. The integrated dryer 300 also includes a VAM 22 which is connected to the process chamber 306 as previously described so that the aerosol 76 is delivered to the process chamber 306 through the aerosol chamber port 320 located beneath a lid 322 and above the process liquid 318 level.

Another of the device embodiments of the invention is a single wafer dryer 400 which is part of a spin processor 402, or the like, described in more detail in FIG. 8. The typical spin processor 402 includes a motor 404 rotatably connected to a spindle 406 and chuck 408 which rotate inside a process bowl 410. In this instance, the chuck 408 is the object holder and the object is a semiconductor wafer 412. The chuck 408 supports the wafer 412 as they rotate. The spin processor 402 provides for generally placing the single wafer dryer 400 in close proximity to the wafer 412. The single wafer dryer 400 includes the previously described VAM 22, the DL management system 24, and aerosol management system 26. The single wafer dryer 400 also includes the aerosol feed tube 94 which terminates in one or more aerosol exit ports like 414, 416, and 418. The aerosol exit port like 414 directs the aerosol 76 generated in the VAM 22 towards a first surface 420 of the wafer 412 to form a drying layer 422 comprising the aerosol 76 across the first surface 420.

If the wafer 412 is loaded and unloaded from above the chuck 408, the aerosol feed tube 94 preferably rotates from the dispense position shown in FIG. 8 to a position beyond the wafer edge 426 so that the aerosol exit port like 414 is no longer over the first surface 420. Alternately, the aerosol feed tube 94 can rotate from the wafer center 424 to the wafer edge 426 while the aerosols 76 is being dispensed. The aerosol feed tube 94 can then remain beyond the wafer edge 426 during a subsequent unload and load of the wafer 412 from above the chuck 408.

During aerosol 76 dispense, the wafer 412 rotates at a low speed, less than about 1000 rpm. A typical aerosol dispense time is between about 5 seconds to about 30 seconds. The thickness of the drying layer 422 across the wafer 412 can be controlled by several factors such as the exhaust velocity in the process bowl 410, the wafer 412 rotation speed, the aerosol 76 dispense rate, the position of the aerosol exit port 414 relative to the wafer center 424 or wafer edge 426, and the like.

The invention controls the thickness of the drying layer 422 from the wafer center 424 to the wafer edge until the drying layer 422 is completely removed. For example, decreasing the exhaust velocity in the process bowl 410 increases the thickness of the drying layer 422 at the wafer center 424 and decreases the thickness of the drying layer 422 at the wafer edge 426. Increasing the exhaust velocity reverses the effect on the drying layer 422. Once the aerosol dispense is complete, the wafer 412 rotates a high speed, more than about 1000 rpm.

Another device embodiment of the invention is a dual-sided, single wafer dryer 428 which is part of a spin processor 402, or the like, illustrated in more detail in FIG. 9. As previously described with reference to FIG. 8, the dual-sided, single wafer dryer 428 has the aerosol feed tube 94 which terminates in the aerosol exit port 414 to direct the aerosol 76 generated in the VAM 22 towards the first surface 420 of the wafer 412 to form a drying layer 422 comprising the aerosol 76 across the first surface 420. The dual-sided, single wafer dryer 428 also includes a second aerosol feed tube 432 which terminates in a second aerosol exit port 434 to direct the aerosol 76 generated in the VAM 22 towards a second surface 430 of the wafer 412 to form a second drying layer 436 comprising the aerosol 76 across the second surface 430. Devices other than the chuck 408 can be used to support the wafer 412 and expose the entire second side 430 of the wafer such as an edge grip and the like which can support the wafer 412 only by contacting the wafer edge 426.

Another single wafer dryer 438 embodiment of the invention is illustrated in more detail in FIG. 10. The single wafer dryer 438 also includes the aerosol feed tube 94 which terminates in the aerosol exit port 80 which directs the aerosol 76 generated in the VAM 22 towards the first surface 420 of the wafer 412 to form a drying layer 422 comprising the aerosol 76. The single wafer dryer 438 includes a second feed tube 440 for dispensing a process liquid 442 from a process liquid container 444 to the process liquid exit port 446. The aerosol exit port 80 is located closer to the center 424 of the wafer than process liquid exit port 446. The single wafer dryer 438 can dispense the process liquid 442 and aerosol 76 relatively simultaneously so that the process liquid 442 passes across the surface 420 of the wafer from the wafer center 424 to the wafer edge 426 followed by the drying layer 422. The movement of the process liquid 442 and drying layer 422 across the surface 420 of the wafer can be controlled leaving the aerosol feed tube 94 and process liquid feed tube 440 in a “static” position and instead varying the rotation of the wafer, exhaust velocity, and the like. Alternately, the aerosol feed tube 94 and process liquid feed tube 440 can rotate “dynamically” across the surface 420 of the wafer.

The embodiments of the invention can be combined with other tools to perform process functions in combination or in sequence with the drying of the object(s). For example, the single wafer dryer 400 shown in FIG. 8 can be combined with a megasonic transducer 450 removably mounted over the wafer 412 and powered by a megasonic generator 452 to clean before drying the wafer 412. A suitable megasonic system is commercially available from Prosys Inc. of Campbell, Calif.

Another device embodiment of the invention is a vertical single wafer dryer 500 shown in more detail in FIGS. 11A and 11B. The dryer 500 includes a single wafer process chamber 502 for holding an object which, in this instance, is a wafer 504 held in a generally vertical position. The process chamber 502 has a top end 506 and bottom end 508 which includes a drain valve 510. The dryer 500 includes the previously described VAM 22, the DL management system 24, and aerosol management system 26. The VAM 22 delivers the aerosol 76 through an aerosol feed tube 94 to the top end 506 and into the empty process chamber 502 so that the aerosol 76 coats the wafer 504.

In FIG. 11B, the wafer 504 is extracted from the process chamber 502 by a conventional wafer handling device (not shown). Preferably, once the aerosol 76 stops dispensing, a heated purge gas 512 is directed into the process chamber 502 while the wafer 504 is still in the process chamber 502 or as the across the wafer 504 near the top end 506 of the process chamber 502 as the wafer 504 is being extracted.

In another embodiment of the invention, the drain valve 510 is closed on the vertical single wafer dryer 500 and the process chamber 502 submerges the wafer 504 in a process or rinse liquid. The VAM 22 delivers the aerosol 76 through an aerosol feed tube 94 to the top end 506 and creates a drying layer 514 on the surface of the process liquid 516 as will be described in more detail below. The drain valve 510 is then opened to drop the level of the process or rinse liquid in the process chamber 502 and allow the drying layer 514 to move across both surfaces of the wafer 504. Alternately, the drain valve 510 can remain closed and the drying layer 514 can move across both surfaces of the wafer 504 by extracting the wafer through the top end 506 of the process chamber. Optionally, a heated purge gas 512 is directed into the process chamber 502 while the wafer 504 is still in the process chamber 502 or as the across the wafer 504 near the top end of the process chamber 502 as the wafer is being extracted.

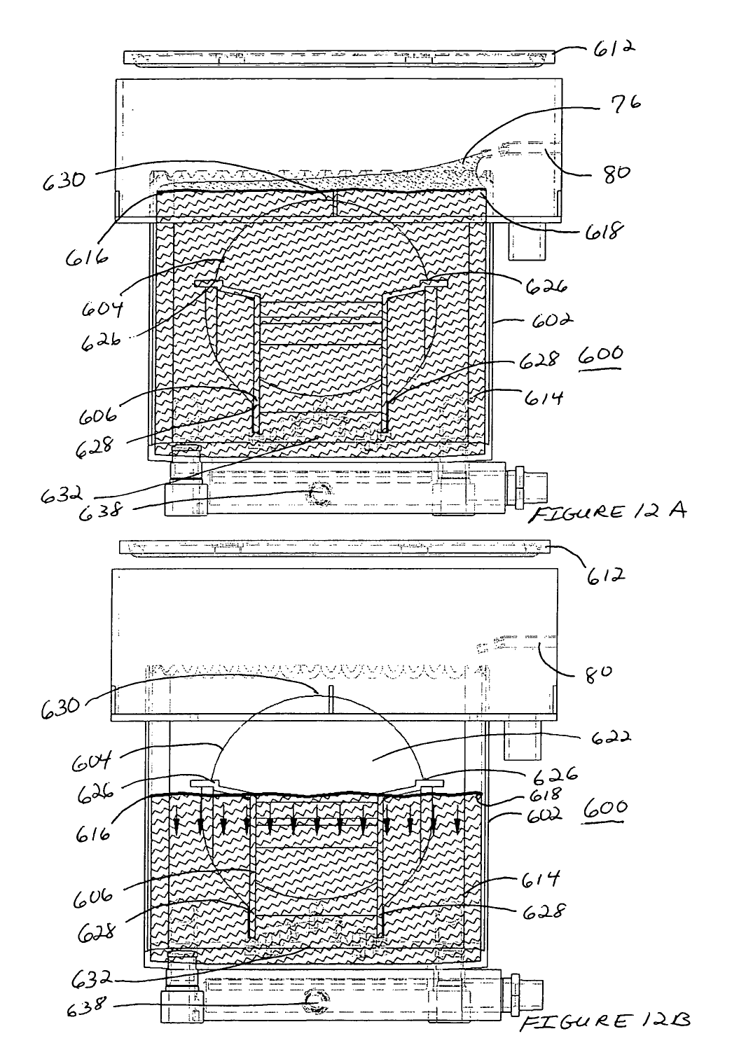

FIGS. 12A, 12B and 12C illustrate the process steps to be taken in one embodiment of the invention. A dryer 600 includes a process chamber 602 holds an object to be dried, in this instance a plurality of semiconductor wafers 604. The wafers 604 are supported in a cassette 606 by an upper set of contact points 626 and a lower set of contact points 628 to define top ends 630 and bottom ends 632 of the wafers. The process chamber 602 has a bottom end 608 and a top end 610 covered by a lid 612. The wafers 604 are submerged in a process liquid 614. The invention can either place the wafers 604 in an empty process chamber 602 and then fill with the process liquid 614 or have the process chamber 602 already filled with the process liquid 614 and then load the wafers 604 into the process chamber 602. The aerosol 76 is directed into the process chamber 602 through the aerosol exit port 80 and creates a drying layer 616 comprising the aerosol 76 on the surface 618 of the process liquid 614.

In FIG. 12B, the process liquid 614 begins a controlled drain through 638 from the process chamber 602 lowering the process liquid surface 618 as indicated by arrows 624. Accordingly, the drying layer 616 moves across the surfaces 620 of the wafers 604 to remove the process liquid 614 from the wafer surfaces 620 and expose an increasing portion 622 of the wafer surfaces 620.

Preferably, the invention uses multiple drain speeds to decrease process time and is made possible by using a variable drain motor 234 as previously illustrated in FIG. 6B. A first or high drain speed is used to start the controlled drain from the top ends 630 of the wafers until the drying layer 616 reaches the upper set of contact points 626. A second or low drain speed is used as the drying layer 616 moves across the upper set of contact points 626 to promote continuity of the process liquid 614 and drying layer 616 and prevent back-splashing of the exposed surface portion 622 in the area near the upper contact points 626. Once the drying layer 616 is past the upper contact points 626, the controlled drain reverts to the first or high drain speed until the drying layer 616 reaches the lower set of contact points 628. The second or low drain speed is used as the drying layer 616 moves across the lower set of contact points 628. Once the drying layer 616 is past the lower contact points 626, the controlled drain reverts to the first or high drain speed until the drying layer 616 reaches the bottom ends 632 of the wafers. After the drying layer 616 reaches the bottom ends 632, a third or dump drain speed can be employed to empty the remainder of the process chamber 602 as quickly as possible. Examples of suitable drain speeds move the drying layer 616 from about 2 mm/sec to about 4 mm/sec for the first or high drain speed, about 0.5 mm/sec for the second or low drain speed, and a dump or the maximum drain speed capable by the drain motor for the third drain speed. The invention includes using more or less than three drain speeds. Rather than a cassette or object holder, it is also possible that the object being dried may have features wherein it is preferred to use multiple drain speeds corresponding to the features.

In FIG. 12C, once the controlled drain is completely past the bottom ends 632 of the wafers and the process chamber 602 is nearly drained, an optional process step of the invention is to direct a purge gas 634 into the process chamber 602 from a manifold 636 in the lid 612. A drain valve 638 is also opened and an air amplifier like 238 illustrated above in FIG. 6A can assist in providing a laminar flow of the purge gas 634 through the process chamber 602 and at least partially out through the drain. Preferably, a purge gas of nitrogen is used at a temperature of about 215 C.°, at a flow of about 100 L/min to about 200 L/min, and for a time period sufficient to evaporate any liquid on the wafers 604 and/or cassette 606. It is desirable to have room temperature nitrogen flowing from the manifold 636 during each step of the process (aerosol dispense and drain) typically at a low flow rate of about 5 L/min.

In one inventive embodiment, dispensing the aerosol 76 into the process chamber 602 stops before the controlled drain. The invention also includes the process steps of dispensing the aerosol 76 into the process chamber 602 throughout the entire, or any portion, of the controlled drain. As will be seen in the examples herein, pulsing the dispense of the aerosol 76 in an ON-OFF-ON-OFF VAM duty cycle during the controlled drain yielded significantly better results than continuously dispensing the aerosol 76 during the entire controlled drain.

Another unique feature and advantage of the invention is that the concentration of the DL 30 can be dynamically controlled while the aerosol 76 is being created which allows for adjusting the thickness of the drying layer 616 while it moves across the surfaces 620 of the wafers 604. It can be desirable to increase the concentration of the aerosol 76 as the drying layer 616 moves across the upper contacts 626 and the lower contacts 628 or other topological features of an object or its holder. The invention increases the concentration of the aerosol 76 by increasing the flow of the carrier gas 72. For example, at a carrier gas flow of 10 L/min about 2.5 ml/min of DL was consumed; at 8 L/min about 2.0 ml/min of DL is consumed; at 6 L/min about 1.0 ml/min of DL is consumed; at 3 L/min about 0.67 ml/min of DL is consumed.

A preferred thickness of the drying layer is less than about 0.5 mm. It is not required that the drying layer remain the same thickness during the entire controlled drain or drying cycle. The invention also includes the use of a drying layer thicker than 0.5 mm.

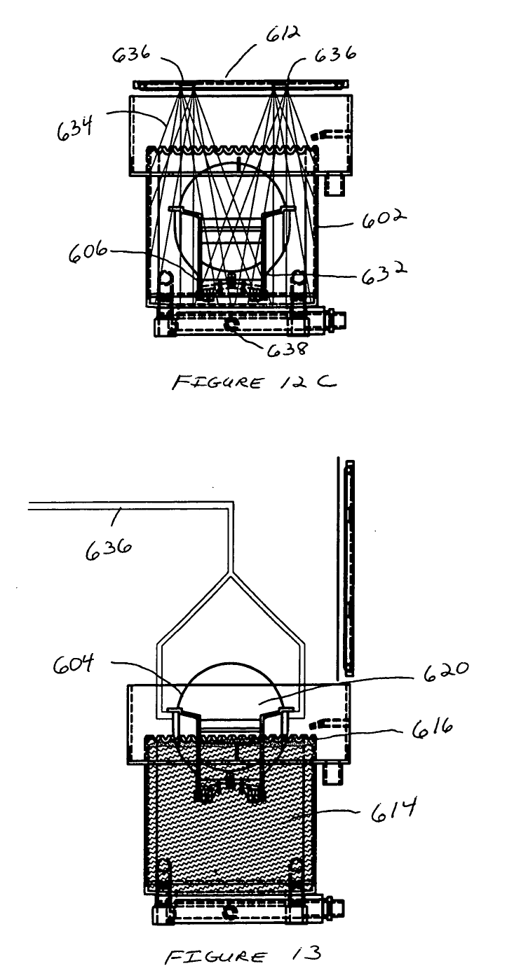

FIG. 13 illustrates another embodiment of the invention wherein the drying layer 616 is passed across the surfaces 620 of the wafers by moving the wafers 604 and keeping the drying layer 616 relatively stationary. In this embodiment, the process liquid 614 is not drained from the process chamber 602. Instead, the cassette 606 holding the wafers 604 is lifted by a mechanism 636 from the process liquid 614 causing the drying layer 616 to pass across the surfaces 620 of the wafers as they emerge from the process liquid 614. Alternately, a combination of lifting the wafers 604 and draining the process liquid 614 can be used to pass the drying layer 616 across the surfaces 620 of the wafers.

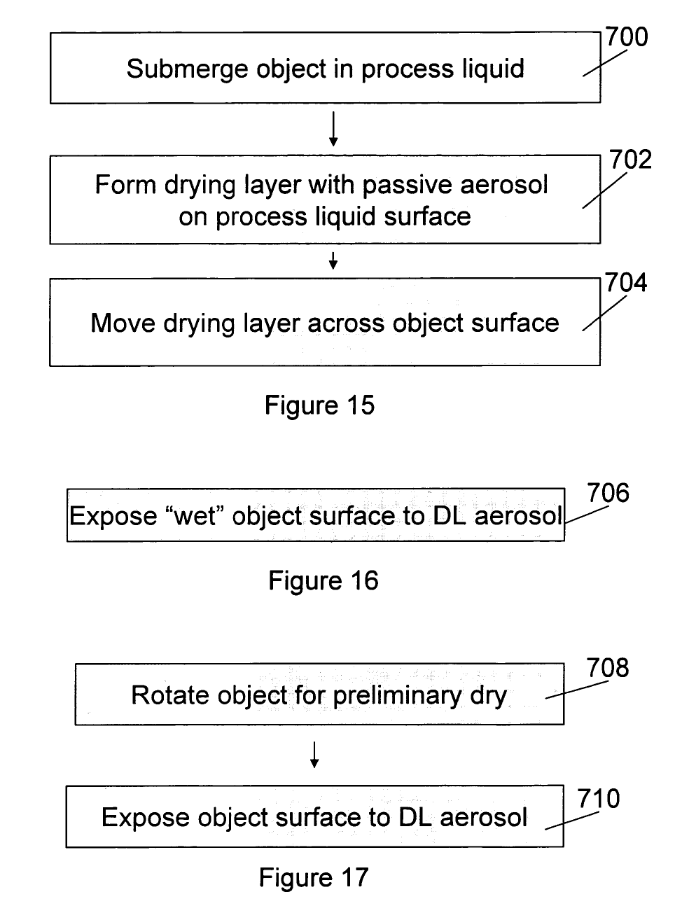

FIG. 15 is a flow chart indicating the process steps to be taken in one embodiment of the invention. In step 700, an object to be dried like the wafers shown in FIGS. 12A, 12B, and 12C, are submerged in a process liquid. Step 702 forms a drying layer by dispensing a passive aerosol over the surface of the process liquid. Step 704 moves the drying layer across the wafer surfaces. Step 704 can be accomplished by dynamically moving the drying layer across the relatively stationary wafer surfaces. Alternately, step 704 can dynamically move the wafer surfaces through a relatively stationary drying layer by not draining the process liquid and lifting the wafers instead. Either a single object or a plurality of objects can be simultaneously moved through the drying layer. The movement of the drying layer can be along a generally vertical axis or a horizontal axis such as by moving a wave of DL across the surface of a horizontally spinning wafer. Optionally, the inventive method includes exposing the wafer surface to a purge gas, preferably heated.

FIG. 16 illustrates a wet-wet-dry process wherein an object such as a wafer undergoes wet processing and is immediately and completely dried to avoid or minimize any wafer surface-air interface. In step 706, the wafer surface is still “wet” with the last process liquid and is immediately exposed to a DL aerosol. Optionally, the wafer surface may then exposed to a heated purge gas like nitrogen or the wafer may be spun at high speed.

FIG. 17 illustrates a wet-dry-wet-dry process wherein an object such as a wafer undergoes wet processing. In step 708, the wafer is spun at a rate and period of time until the wafer has undergone a preliminary dry. For example, the wafer is spun at 1000 to 6000 rpm for a period of time between about 5 to 30 seconds. Due to the hydrodynamic boundary layer, only the higher portions of the water layer are removed by centrifugal spin drying and there is still a portion of the water closest to the surface of the wafer that is more difficult to remove. Even though the wafer may be considered “dry” there are still some residual moisture remaining on the wafer surface. In step 710, the wafer surface is exposed to a DL aerosol for a complete dry. Optionally, the wafer surface may then exposed to a heated purge gas like nitrogen or the wafer may be spun at high speed. It is possible during step 708 that the preliminary dry may expose portions of the wafer surface to the air or other gas present in the process bowl. Some processes may find it desirable to avoid or minimize the wafer surface air interface that can result from the preliminary dry step 708.

Another unique feature and advantage of the invention is the ability to dynamically control the thickness of the drying layer and its movement across the surface of an object during the drying process. Each of the following factors can be independently adjusted during the drying process to affect the thickness and position of the DL across the object surface: Concentration of the DL aerosol, exhaust velocity, rotational velocity and acceleration, and traverse movement of the aerosol dispense port.

### EXAMPLES

A stand alone dryer of the type described herein, was used in a semiconductor facility to dry one cassette of 25 wafers @ 200 mm per each production run. These were actual production wafers that had been subjected to various semiconductor processes including a CMP polishing, megasonic cleaning, and chemical processing using SC1, SC2, and dilute HF and the like. The stand alone dryer was loaded and unloaded manually in a class 100 clean room using isopropanol as the drying liquid. A purge gas of nitrogen at about 215 C.° was used for about 250 to 300 seconds after the process chamber was drained. The measurements were performed with a WIS CR-80 particle detection instrument. With over one thousand consecutive production runs, the results were particle neutral at 130 nm. The size of the process chamber was about 15 inches by 12 inches and contained about 8.5 gallons of de-ionized water when filled. Typically, the VAM dispensed aerosol for less than about four minutes at a rate of 0.5 ml/min for a total isopropanol usage of less than about 3 ml per production run.

An integrated dryer of the type described herein, was used in a semiconductor fab to dry two cassettes of 25 wafers @ 200 mm per each production run. The integrated dryer was loaded and unloaded robotically in a mini-environment using isopropanol as the drying liquid. A purge gas of nitrogen at about 215 C.° was used for about 250 to 300 seconds after the process chamber was drained. The measurements were performed with a Tencor SP-1 particle detection instrument. The results were particle neutral at 100 nm.

Several different aerosol dispense times were tested using a stand alone dryer of the type described herein in a semiconductor facility to dry one cassette of 25 wafers @ 200 mm per each test run. The stand alone dryer was loaded and unloaded manually in a class 100 clean room using isopropanol as the drying liquid. The measurements were performed with a WIS CR-80 particle detection instrument. The aerosol dispense was allowed to run from about 10 seconds to about 90 seconds on different test runs to create the IPA drying layer before using a controlled drain. IPA build times as short as 10 seconds were successful in completing the dry with 130 nm particle neutral performance. A purge gas of nitrogen at about 215 C.° was used for about 250 to 300 seconds after the process chamber was drained. The aerosol dispense (AD) periods were varied: AD only prior to controlled drain; AD prior to and during entire controlled drain; AD prior to and during last half of controlled drain; AD prior to and then pulsing ON-OFF during the controlled drain for various time periods ranging from 10 to 20 seconds. The AD prior to and during the entire controlled drain appeared to give the worst results with some particle contamination at 160 nm. The AD prior to and then pulsing at 15 second periods of OFF and ON was one of the better particle neutral performances at 130 nm. Any combination of these AD periods is included in the invention.

A stand alone dryer of the type described herein was used to dry a Micro-Electro-Mechanical-System (MEMS) device using the approximate parameters described in the first example. The MEMS device had a high aspect ratio of at least 80 to 1 and the invention was successful in drying the deep topology.

The invention has been used to also successfully dry other objects such as wafers thinned to less than about 40 microns; wafers spaced apart at less than about 0.125 inches; fragile wafers made of GaAs and other III-V, II-VI compound semiconductor materials; quartz, sapphire, and other high purity materials; hydrophobic surfaces; optics; flat panel displays; and the like.

While particular embodiments and applications of the invention have been illustrated and described, it is to be understood that the invention is not limited to the precise construction disclosed herein and that various modifications, changes, and variations will be apparent to those skilled in the art may be made in the arrangement, operation, and details of construction of the invention disclosed herein without departing from the spirit and scope of the invention as defined in the appended claims.

What is claimed is:

1. A method of drying an object comprising:
submerging an object having a object surface in a process liquid having a process liquid surface;
creating a passive aerosol;
exposing the process liquid surface to the passive aerosol of a drying liquid to form a drying layer on the process liquid surface; and
moving the drying layer across the object surface.

2. The method of claim 1 wherein the step of creating a passive aerosol includes:
rapidly decreasing the pressure of a carrier gas stream;
entraining the drying liquid into the pressure means to create a venturi flow mixture of the carrier gas stream and the drying liquid; and
striking the venturi flow mixture into a primary baffle to create the passive aerosol.

3. The method of claim 2 wherein the step of creating a passive aerosol further includes:
directing the passive aerosol through a secondary baffle whereby the mass median aerodynamic diameter of the passive aerosol is decreased.

4. The method of claim 1 wherein the method further includes delivering a heated purge gas across the exposed surface of the object after exposure of the surface to the passive aerosol.

5. The method of claim 1 wherein the moving step includes draining the process liquid causing the drying layer to move across the surface of the object.

6. The method of claim 1 wherein the moving step includes moving the object through the surface of the process liquid causing the drying layer to move across the surface of the object.

7. The method of claim 1 wherein the drying liquid is selected from the group consisting of isopropanol, HFE, methanol, ethanol, acetone, tetrahydrofuran, perfluorohexane, ether, hexane, and ethylated hydrofluoroether.

8. The method of claim 1 wherein the object to be dried is selected from the group consisting of a single wafer of semiconductor material, a plurality of wafers of semiconductor material, a quartz substrate, a sapphire substrate, a hydrophobic surface substrate, an optic, a flat panel display, and a MEMS device.

9. A method of drying an object comprising:
submerging an object having a object surface in a process liquid having a process liquid surface;
creating an aerosol having a mass mean aerodynamic diameter of less than 50 microns;
exposing the process liquid surface to the aerosol to form a drying layer on the process liquid surface; and
moving the drying liquid layer across the object surface.

10. The method of claim 9 wherein the aerosol has a mass median aerodynamic diameter of 10 microns or less.

11. The method of claim 9 wherein the concentration of the aerosol is between about 0.6 ml of drying liquid per 3 L of a carrier gas to about 3 ml of drying liquid per 10 L of carrier gas, the carrier gas is nitrogen.

12. The method of claim 9 wherein the thickness of the drying layer is less than about 0.5 mm.

13. The method of claim 9 wherein the method further includes delivering a purge gas across the surface of the object after the object has been exposed to the aerosol.

14. The method of claim 9 wherein the moving step includes draining the process liquid causing the drying layer to move across the surface of the object.

15. The method of claim 9 wherein the moving step includes moving the object through the surface of the process liquid causing the drying layer to move across the surface of the object.

16. The method of claim 9 wherein the drying liquid is selected from the group consisting of isopropanol, HFE, methanol, ethanol, acetone, tetrahydrofuran, perfluorohexane, ether, hexane, and ethylated hydrofluoroether.

17. A method of drying an object comprising:
submerging an object having a object surface in a process liquid having a process liquid surface;
creating an aerosol of a drying liquid with no expenditure of energy;
exposing the process liquid surface to the aerosol to form a drying liquid layer on the process liquid surface; and
moving the drying liquid layer across the object surface.

18. The method of claim 17 wherein the method includes using less than 3 ml of drying liquid to dry 25 objects each having two sides of 200 mm in diameter.

19. The method of claim 17 wherein the drying liquid is selected from the group consisting of isopropanol, HFE, methanol, ethanol, acetone, tetrahydrofuran, perfluorohexane, ether, hexane, and ethylated hydrofluoroether.

20. A method of drying an object having an exposed surface comprising:
creating an aerosol from a drying liquid; and
delivering the aerosol to the exposed surface of the object.

21. The method of claim 20 wherein the method further includes rotating the object about a central axis.

22. The method of claim 20 wherein the drying liquid is selected from the group consisting of isopropanol, HFE, methanol, ethanol, acetone, tetrahydrofuran, perfluorohexane, ether, hexane, and ethylated hydrofluoroether.
